(19) United States
(12) Patent Application Publication
(10) Pub. No.: US 2014/0236179 A1
(43) Pub. Date: Aug. 21, 2014

(54) Medically Inspired Tick Removal Instrument

(71) Applicants: Robert E Henson, Piedmont, MO (US); John Henson, JR., Piedmont, MO (US)

(72) Inventors: John Henson, JR., Piedmont, MO (US); Robert E Henson, Piedmont, MO (US)

(21) Appl. No.: 14/109,778

(22) Filed: Dec. 17, 2013

Related U.S. Application Data
(60) Provisional application No. 61/765,660, filed on Feb. 15, 2013.

(51) Int. Cl. A61B 17/50

(52) U.S. Cl. CPC A61B 17/50
USPC 606/131

(57) ABSTRACT

It is of utmost importance an embedded tick is removed promptly after attachment, without causing distress to prevent possible infection from harmful diseases. Problems are created when the cheliceral digits (ref #17) and barbed hypostome (ref #18) are disassociated and left within the host's dermis. The Medically Inspired Tick Removal Instrument is able to remove the entire tick. The tick engaging portion includes a “u-groove” (ref #8) and an anterior framework (ref #5) which close upon each other to trap the tick. The triangle-shaped base (ref #9) includes a specifically designed hemispherically protruding ball (ref #7) which causes it to release its grip from within the host's dermis. Ergonomic thumb and finger locators (ref #11 & 12) facilitate the use of the tool in a comfortable position suitable for use by either hand. The tick is easily removed by a gentle lifting motion of the tool.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

### Field of Search

CLASS 128 SURGERY

CLASS 606 SURGERY (This Class, 606 is considered to be an integral part of Class 128)
1 INSTRUMENTS
131 . . . Means for removal of skin or material there from
210 . . . Tweezers

CLASS 294 HANDLING: HAND AND HOIST-LINE IMPLEMENTS
294-1
99.1 . . . Resilient jaws
99.2 . . . Hand-held
86.28 . . . Outwardly biased grappling means

### CROSS-REFERENCE ART COLLECTIONS

902 GRIPPING ELEMENT
897 MISCELLANEOUS
898 . . . Methods

CLASS D24, MEDICAL AND LABORATORY EQUIPMENT
133 . . . Hand manipulated tool or instrument (23)
143 . . . Clamp or forceps (28)
147 . . . Manually driven (32)
149 . . . Opposed

Field of Search Classes
219/227, 219/233, 219/229, 294/99.2, 294/100, 606/206, 606/131, 606/133, 606/264, 606/27, 606/29, 606/42, 606/210, 606/211, 362/157, 362/204, 362/202, 362/208, 362/119, 43/134

1. Field of the Invention

Medically Inspired Tick Removal Instrument relates to devices for removing foreign objects from the surface of the skin; in particular, removing ticks from the dermis of animals and humans alike. The present invention relates generally to the field of surgical instruments and devices for removing a parasite, namely a tick from a host's (human or animal) dermis.

2. Description of the Prior Art

In recent years, there has been an alarming increase in the incidence and geographical range of tick-born disease. Both humans and animals are affected by a number of infectious agents carried in and transmitted by ticks, such as Rocky Mountain Spotted Fever and Lyme Disease.

When a tick attaches to a human or an animal host, it not only inserts a portion of its gnathasoma (cheliceral digits and hypostome) into the host to reach the blood supply, it also secretes an adherent cement into and onto the skin of the host. This cement helps prevent the tick from becoming detached from the host. After the tick attaches to the host, infectious agents within the tick may be transmitted to the host by the tick during its feeding cycle.

Infectious agents begin transmission in as few as four (4) hours after host attachment. The sooner the parasite is removed, the lower the risk of infectious agents being transmitted to the host. Therefore, the best method of protecting the host from any diseases carried by the tick is a rapid, effective, and complete removal of the tick from the host.

In recent generations many methods have been expounded as the proper way to remove a tick. These include but are not limited to: paint the tick and bite area with nail polish, petroleum jelly or kerosene in an attempt to suffocate the tick. It was postulated if the tick could not breathe, it would let go and back itself out. Burning the tick out with matches was also a popular method in attempting to remove a tick. The expansion of science has taught one that these methods were merely folklore remedies. We learned that the tick was simply completing its feeding cycle and releasing its grip on the host when done.

These crude methods were not then and are currently not effective. In fact, they often exacerbate the tick until it releases its gut fluids into the host. The application of heat or above-related substances to the tick bite site is painful and can be dangerous especially when working with a child or pet. It can also be unfavorable in causing an allergic reaction in some. Science has taught one about the presence of dangerous substances in the tick's gut fluids. It is here that the disease-causing agents are contained. Lyme Disease and Rocky Mountain Spotted Fever are two examples.

Other recommendations include twisting the tick around in a circle until it is no longer attached to the host. This recommendation is ineffective in preventing disease transmission because the motion severs connection between the tick's gnathasoma and podosoma. The cheliceral digits, hypostome and adhering cement are left in/on the host. The tick is often squeezed during attempted removal. This squeezing action forces gut fluids from within the tick into the host. These fluids may contain harmful or even disease-causing bacteria. Twisting the tick most certainly serves to separate the tick's podosoma from the cheliceral digits and barbed hypostome while leaving said mouth parts within the host.

The cheliceral digits and barbed hypostome are only a few hundredths of a millimeter in diameter. Ticks have a well defined division between the gnathasoma and the podosoma. The tick excretes a thick substance in which it effectively cements itself within and onto the host's dermis. This makes the risk of separation even greater.

A more specific object of previous inventions is to provide a composition which aids in the removal of a tick from its host. Chemical agents can potentially irritate the tick and thereby stimulate it to transmit its gut fluids possibly containing infectious agents to the host. The host can have an adverse reaction to the chemical agents applied while attempting tick removal. Chemical agents that do stimulate the tick to detach from the host will not reliably remove the adherent cement excreted by the tick. It problematically remains in and on the dermis of the host after detachment. The cement leaves a wound in the dermis of the host. The adherent cement may carry infectious agents itself. It is not yet known. Regardless, the risk of infection is greatly increased.

Chemical agents must be available for use within a short period after the tick attaches. It is often not possible to bring a person or animal to a supply of the chemical agent quickly enough. Alternatively, it is unlikely that people involved in outdoor recreation will carry a supply of a tick removal agent with them at all times. Some people are opposed to the practice of chemical agents. Chemical agents when used on children or animals may sting, burn or otherwise be less than appealing to the host. The host is provoked to react violently against peaceful treatment.

The CDC's current recommended method for removal of an imbedded tick is to lift a tick out of the host with tweezers. One is instructed to secure the tweezers around the tick, as close to the host's skin as possible. Pull with a steady, constant motion.

Ideally, the best method to prevent transmission of disease from the tick to the host is the complete removal of the tick including all cheliceral digits and barbed hypostome which have been inserted into the host and the adherent cement by mechanical means. A problem inherent in mechanical a system is that care must be exercised to avoid squeezing or crushing the body of the tick during removal. Saliva or gut contents which may contain infectious agents can be regurgitated into the human or animal host.

In order to effect mechanical removal of the tick from the host, a number of prior art forceps and tweezers have been developed. One problem with forceps and tweezers of the prior art is that they rely on pressure against the body of the tick in order to obtain an adequate purchase for removal. In other words, the prior inventions clamp the body of the tick like a pair of pliers. Any squeezing of the body of the tick may cause gut/body fluids of the tick to be expelled into or onto the host. This inherently increases the likelihood of exposure to dangerous elements from within the tick.

Another problem with the prior art tick removal forceps is that the tips are designed to clamp solidly together in continuous full surface contact. This makes removal of ticks in hair bearing areas or in the invitingly warm folds and creases of the host's dermis difficult. The host's dermis may be pinched or the hair may be pulled. Discomfort to the host makes removal of the tick more traumatic, particularly if the host is a child or an animal.

Some prior inventions have a sharp edge that is to be placed around the tick's gnathasoma or between the cheliceral digits, barbed hypostome and the alloscutum. This sharp edge has been proven in independent studies to frequently divide the two body areas leaving the cheliceral digits and barbed hypostome secured within the host's dermis.

3. Background of the Prior Art

Ordinary tweezers, forceps and other prior inventions are not suitable for properly removing embedded ticks from the dermis of humans or animals. Attempting removal of a tick by merely grasping the body and pulling (or twisting, as some prescribe for removal) nearly ensures leaving the cheliceral digits and barbed hypostome in the host's dermis. Increasing the likelihood of infection occurs when the gnathasoma is separated from the podosoma of the tick. The host is exposed to conditions such as Rocky Mountain Spotted Fever, Lyme Disease and other conditions yet to be adequately understood.

Squeezing force can only be regulated in a crude fashion. This is a distinct disadvantage of ordinary tweezers, forceps and other prior inventions. These prior art pieces are closed by squeezing with one's fingers the apparatus. The squeezing force applied can easily be too great forcing the tick to regurgitate its fluids into the victim's skin. If the embedded portion of the gnathasoma is too firmly attached within the skin, the result is customarily decapitation of the parasite. Attempting to grasp the head while still embedded in the victim's skin poses further difficulties. There is both harm and risk in severing or rendering the gnathasoma from the podosoma during attempts to remove a feeding tick from its host. It is common knowledge and expounded upon in the prior art.

Some of the art is directed to professionals who can justify more expensive equipment. Many of the previous inventions are available to the casual user, (e.g. parents, pet owners, hunters or farmers) by using cheaper, simpler and more readily available materials. The present invention can be used by professionals and lay people alike. The previous art's lack of recognition of the ergonomic benefits and improved performance achievable through design innovations which combine the sensitivity available in natural bi-dexterous hand placement with complimentary structure which is a task-specific extension of the user's hand has impeded the natural progression of the invention process.

### BRIEF SUMMARY OF THE INVENTION

The main purpose of the tool is to provide a simple means for the complete, intact removal of a tick or similar parasite which has its head and/or jaws embedded in the skin of a host animal or person. Medically Inspired Tick Removal Instrument is a bi-dexterous tool which can be readily grasped, manipulated and softly compressed by either hand. Locators are provided near the posterior of the handles for thumb and forefinger positioning and actuation.

The invention provides a hand tool that employs a natural, palm-down, ergonomic-benefiting hand position and gripping motion closely simulating the motions that might be used to extract the tick with one's own thumb and forefingers. While maintaining a substantially unobstructed view of the tick, one is able to manipulate the tool with one hand without excessive arm or wrist motion,

Further, there is a minimal parts count. Medically Inspired Tick Removal Instrument may be molded as a single piece tool. The present invention can be made out of a number of materials such as stainless steel, metal alloy or plastic.

Medically Inspired Tick Removal Instrument's precisely designed head and exacting location of the hemispherical protruding ball safely removes the cheliceral digits and barbed hypostome from within the host. Lack of provocation to the parasite helps to prevent contamination of the host by the fluids contained within the gut fluids of the tick. When Medically Inspired Tick Removal Instrument is used properly, according to provided instructions, the cheliceral digits and barbed hypostome is removed every time. Independent testing has shown that there is no invention to this date that can adequately perform to this standard. One is able to see the remaining cementing fluids attached to the tick's cheliceral digits and barbed hypostome when the tick is excised from within the host's dermis.

Medically Inspired Tick Removal Instrument is configured and sized to substantially enclose the body of the tick when the invention is in the closed position. The apparatus is designed to surround the cheliceral digits and barbed hypostome of the tick when the arms are in the closed position. Medically Inspired Tick Removal Instrument is designed to ensure the tick is left intact for proper disposal or investigation. The active portions of the invention are mounted on the opposite end of the opposing arms.

### BRIEF DESCRIPTION OF THE SEVERAL VIEWS OF THE DRAWINGS

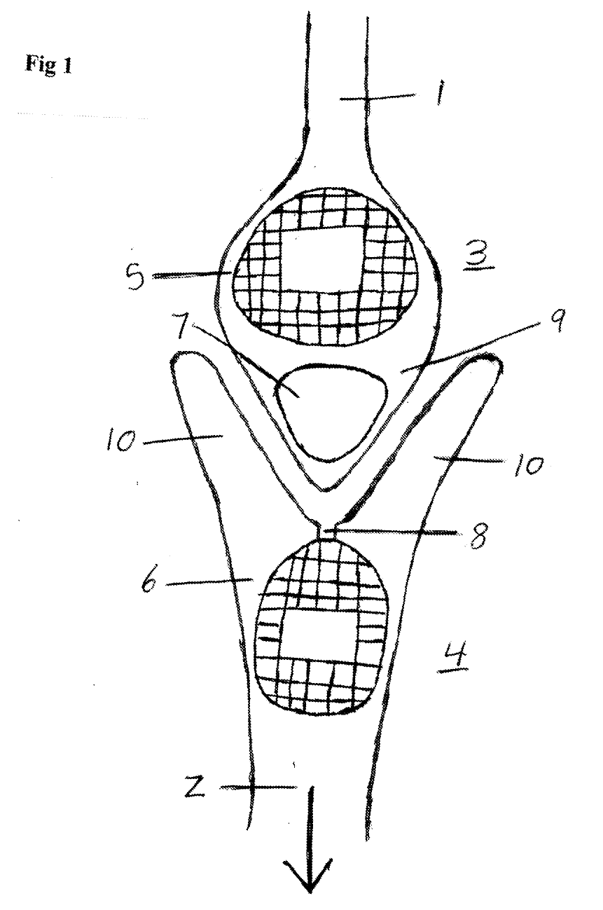

Drawing 1/5 FIG. 1 Inside View Open position View of the interior of part 1 upper arm and part 2 lower arm in an open position

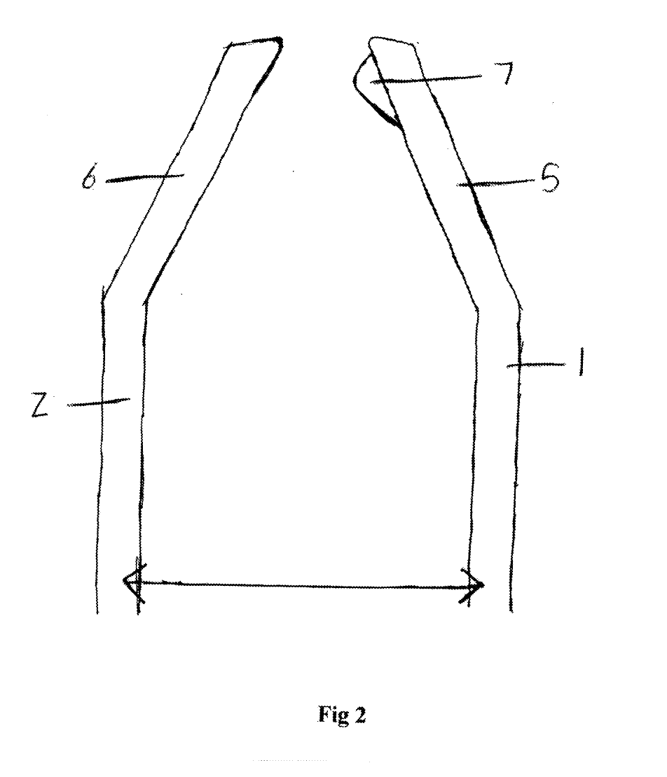

Drawing 2/5 FIG. 2 Side View Open position A side elevation of Medically Inspired Tick Removal Instrument part 1 upper arm and part 2 lower arm in an open position

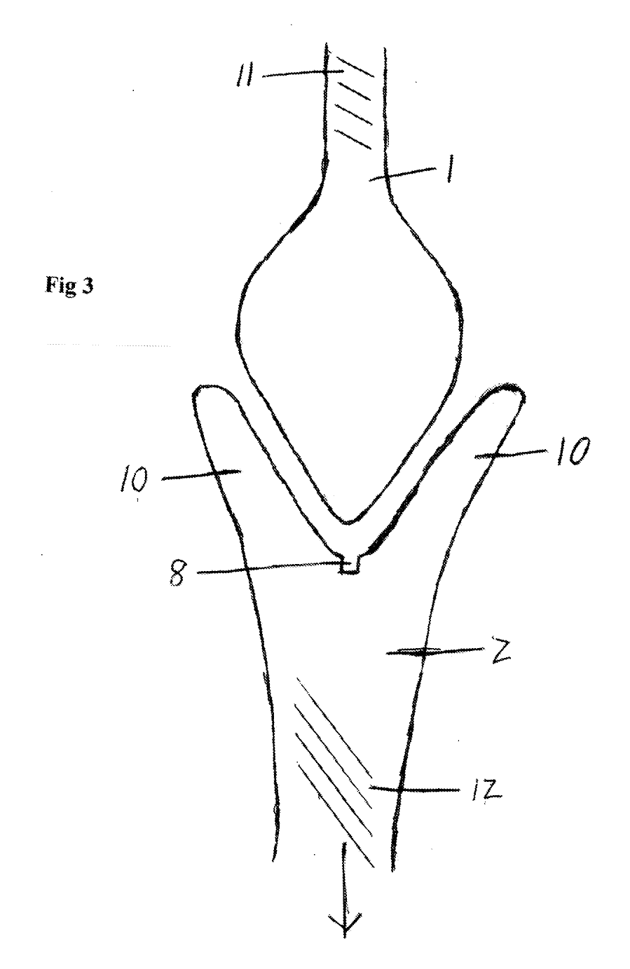

Drawing 3/5 FIG. 3 Outside View Open position View of the exterior of part 1 upper arm and part 2 lower arm in an open position

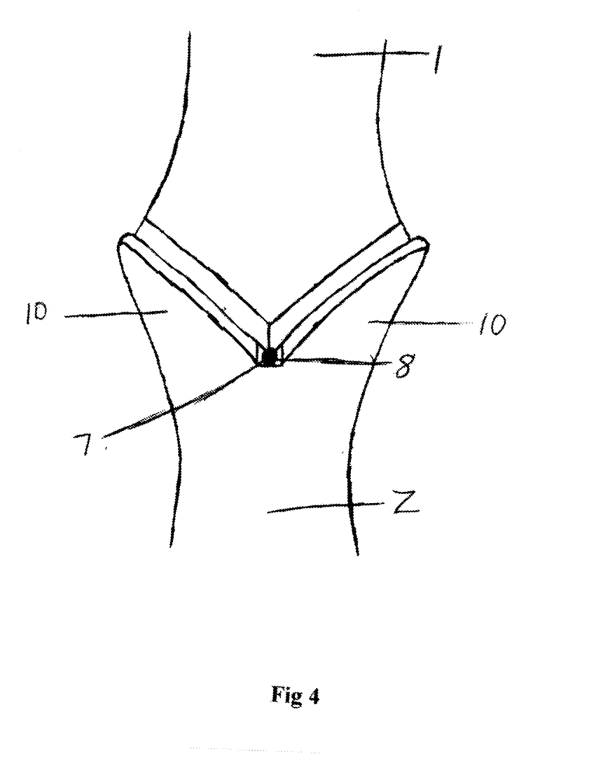

Drawing 4/5 FIG. 4 End View Closed position Closing of the gripping points takes place

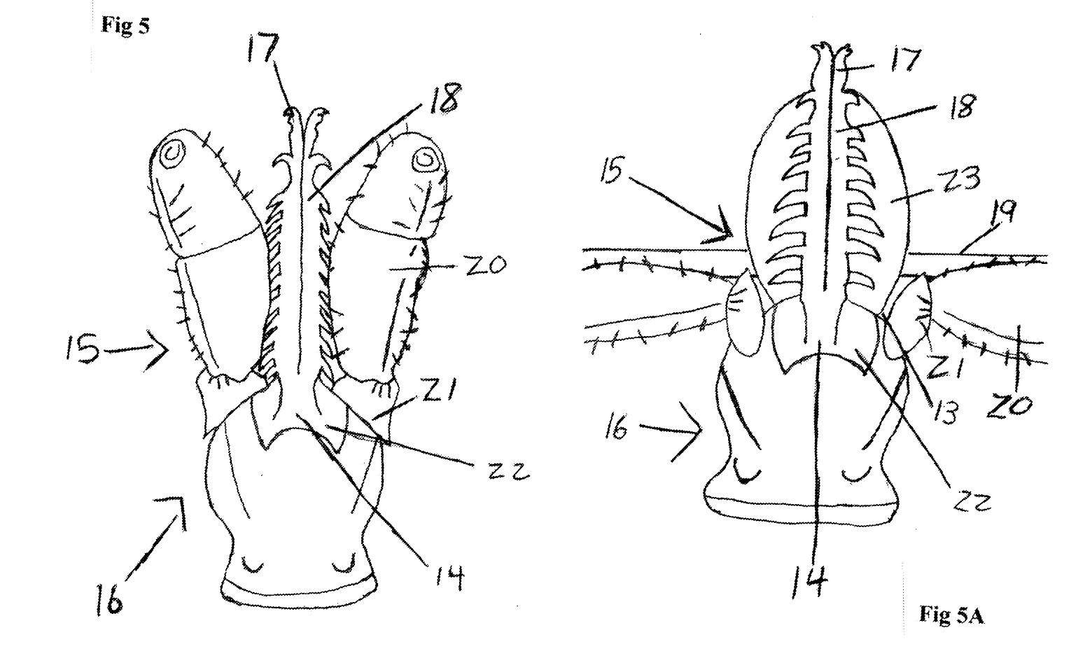

Drawing 5/5 FIG. 5 Close-up sketch of hard-bodied tick such as found in Class Ixodes Features body sections gnathasoma and podosoma in relaxed position

Drawing 5/5 FIG. 5A Close-up sketch of embedded hard-bodied tick such as found in Class Ixodes Features body sections gnathasoma portions of which are embedded and podosoma in active feeding position

### DETAILED DESCRIPTION OF THE INVENTION

The height of the upper and lower arms in a closed position is 3.175 mm. Width of closed Medically Inspired Tick Removal Instrument (ref #10) is 2.381 mm. The thickness of the distal end of Medically Inspired Tick Removal Instrument (ref #1 & 2) is 1.051 mm+/−0.051 mm. The thickness of Medically Inspired Tick Removal Instrument near the holding cavities (ref #3 & 4) are 0.457 mm+/−0.051 mm. The thickness chosen for Medically Inspired Tick Removal Instrument arms is 0.381 mm, permitting controlled bending and flexing throughout the anterior frameworks (ref #5 & 6) section as desired by the user.

FIGS. 1, 2, 3, and 4 are constructed according to the present invention. It relates to the manual removal of live ticks from the host whose cheliceral digits (ref #17) and barbed hypostome (ref #18) are embedded within the host. The gnathasoma (ref #15) of the tick is smaller than the podosoma (ref #16). The podosoma of the tick may or may not be engorged depending on which stage of its feeding cycle the tick is in.

FIGS. 5 & 5A are sketches of a representative hard-bodied tick such as in the Class Ixodes. The body portion names are generalized to include names currently accepted by professionals in the field.

We intend to protect our invention, Medically Inspired Tick Removal Instrument, by patenting it. Medically Inspired Tick Removal Instrument is the only tool which reliably removes the cheliceral digits (ref #17) and barbed hypostome (ref #18) from within the host's dermis (ref #19). When this instrument is used in the removal process, the chances of regurgitation of body fluids that may contain harmful bacteria or diseases are mitigated. The tick is not squeezed nor divided into separate pieces. Medically Inspired Tick Removal Instrument is the only invention that can perform this.

Medically Inspired Tick Removal Instrument is intended to be made of plastic, stainless steel or a metal alloy. It will be injection-molded in one piece. A stainless steel or metal alloy model would be useful when sterilization prior to each usage is required as in a medical office or in usage by an outdoors person, farmer or pet owner. The plastic form of Medically Inspired Tick Removal Instrument would be useful in a medical or veterinary office where one-time usage would be the best course of action.

Medically Inspired Tick Removal Instrument has part 1, upper arm (ref #1) and part 2, lower arm (ref #2). Medically Inspired Tick Removal Instrument consists of two equal length arms attached by a conjoined handle. The entire length of the instrument is intended to be 5.5 cm. Further instrument parts are as follows:

{1} Part 1 Upper Arm The upper arm of Medically Inspired Tick Removal Instrument's handle is to be horizontally flat at 90°. The arm maintains a width of 0.381 mm throughout and is 3.5 cm long. Upper arm (ref #1) is intended to be conjoined to part 2 lower arm (ref #2).

{3} Upper Holding Cavity The central location of Part 1 (ref #3) is machined 0.481 mm wide, 0.152 mm deep and 0.438 long to form a cavity. The upper holding cavity (ref #3) is located 0.352 mm to the posterior of the distal end of the instrument containing the single rounded projection (ref #5) and anterior to the handle (ref #1) of Medically Inspired Tick Removal Instrument. This concavity is included to contain the entire tick during removal process. The lower holding cavity is included within the anterior framework (ref #6) and its purpose is to contain the entire tick during removal process.

{5} Anterior Framework The anterior framework is connected to upper arm handle (ref #1). This downward yet slightly inward pointing rounded end (ref #5) extends at a 25° angle from handle (ref #1). This framework contains the hemispherical protruding ball (ref #7) and the upper holding cavity (ref #3). The width of the framework fluctuates. It decreases from 0.635 at the widest point around upper holding cavity (ref #3) to 0.152 mm at the most anterior point of the hemispherical protruding ball (ref #7). It decreases from the widest point to 0.381 mm where it meets the handle (ref #1). The rounded tip is machined such as to prevent it from entering the u-shaped channel (ref #8) during actuation of Medically Inspired Tick Removal Instrument. The entire section measures 2 cm long.

{7} The hemispherical protruding ball is located within the anterior framework (ref #5). It is a calculated placement of the same material Medically Inspired Tick Removal Instrument is made of. The hemispherical protruding ball resides on its platform (ref #9). The hemispherical protruding ball begins 0.252 mm back from the tip of the platform (ref #9), extends 0.508 mm and is 0.457 mm thick +/−0.051 mm. The width of the hemispherical protruding ball is 0.381 mm wide nearest the upper holding cavity (ref #3) and gently narrows within its platform (ref #9).

The purpose of the hemispherical protruding ball (ref #7) is to engage the back of the tick's alloscutum (ref #22) during removal from the host at the dorsal contact point (ref #14), It pierces the tick's alloscutum (ref #22) and touches the interior of Medically Inspired Tick Removal Instrument part 2 lower arm where the lower holding cavity (ref #4) and the u-shaped mouthparts channel (ref #8) meet.

{9} This gentle triangle-shaped base contains the hemispherical protruding ball (ref #7). The base is located in the interior portion of the upper arm part 1 rounded tip (ref #5).

{11} An ergonomic thumb and finger locator is on the outer surface of the upper arm. Cross hatches machined into the surface of the instrument provide a placement guide and firm gripping surface for the user. Indication that this is the “top” arm is given here.

{2} Part 2 Lower Arm The lower arm of Medically Inspired Tick Removal Instrument's handle is to be horizontally flat at 90°. The arm maintains a width of 0.381 mm throughout and is 3.5 cm long. Lower arm (ref #2) is intended to be conjoined to Part 1 Upper Arm (ref #1).

{4} Lower Holding Cavity The central location of Part 2 (ref #4) is machined 0.438 mm wide, 0.152 mm deep and 0.481 long to form a cavity. The lower holding cavity (ref #4) is located 0.352 mm to the posterior of the distal end of the instrument containing the dual rounded projections (ref #10) and anterior to the handle (ref #2) of Medically Inspired Tick Removal Instrument. The lower holding cavity is included within the anterior framework (ref #6) and its purpose is to contain the entire tick during removal process.

{6} Anterior Framework The anterior framework is connected to lower arm handle (ref #2). This upward yet slightly inward-pointing section (ref #6) extends at a 25° angle from the handle (ref #2). The framework contains dual, rounded projections (ref #10) and the lower holding cavity (ref #4). The width of the framework increases from 0.461 mm posterior to the lower holding cavity (ref #4) to 0.598 mm at the location of the mouthparts channel (ref #8). The entire section measures 2 cm long.

{8} Mouthparts Channel This small square portion of Medically Inspired Tick Removal Instrument is where the tick's mouthparts, chericeral digits (ref #17) and barbed hypostome (ref # 18) are channeled during the removal process. The u-shaped opening meets the lower arm holding cavity (ref #4) and is located anterior to said cavity. The mouthpart channel is located between the anterior dual, rounded projections (ref #10). The channel is created by a purposeful gap of instrument material measuring 0.152 mm²+/−0.025 mm.

{10} Dual, Rounded Projections The dual, rounded projections are located at the farthest anterior point of the lower framework (ref #6). The projections continue at an additional 10° angle upward from the frame. The projections gently part from one another forming a vee. Each projection is 0.633 mm long and 0.356 mm wide with a gentle narrowing at the tips. Each rounded projection is 0.297 mm thick to aid in scooping under the tick. These projections (ref #10) make contact with the feeding tick (ref #13) and provide leverage for pulling the tick up and away from the host at its insertion site.

{12} An ergonomic thumb and finger locator is on the outer surface of the lower arm, part 2 of Medically Inspired Tick Removal Instrument. Cross hatches machined into the surface of the instrument provide a placement guide and firm gripping surface for the user. Indication that this is the “bottom” arm is given here.

The tick as it is described and sketched herein contains generalities as to body sections of Class Ixodes as currently accepted by professionals in the field.

{13} Forward Contact Points When Medically Inspired Tick Removal Instrument is applied, the lower arm (ref #2) is guided under the tick until the dual, rounded projections (ref #10) scoop into positions anterior to the tick's podosoma (ref #16). The dual, rounded projections (ref #10) come into contact with the tick at the forward contact points (ref #13) to leverage the tick up and away from the host's dermis (ref #20).

{14} Dorsal Contact Point the dorsal contact point is located on tick's alloscutum (ref #22). When Medically Inspired Tick Removal Instrument is softly compressed around the tick during actuation, the hemispherical protruding ball (ref #7) pierces the tick's body at the dorsal contact point (ref #14). It continues through completely to underneath side of the tick.

{15} Gnathasoma A tick's body is generalized into two sections. The anterior portion of the tick's body is referred to as the gnathasoma. Includes cheliceral digits (ref #17), barbed hypostome (ref #18), palps (ref #20), and anterior spurs (ref #21).

{16} Podosoma A tick's posterior portion is generalized into the term Podosoma or what one might think of as the body. The tick's alloscutum (ref #22) is within the podosoma (ref #16).

{17} Cheliceral Digits The chewing portion of the tick is called the cheliceral digits. The cheliceral digits are located on the most anterior portion of the tick at the end of the barbed hypostome (ref #18). Their purpose is to open the wound in the host's dermis (ref #19). The digits continue to chew into the layers until the barbed hypostome (ref #18) is in prime feeding position.

{18} Barbed Hypostome After the cheliceral digits (ref #17) have opened the dermis layers (ref #19) of the host, the barbed hypostome helps to widen the wound. The barbed hypostome (ref #18) has rear-pointing, hooked barbs on either side which helps anchor the tick in position.

{19} Dermis Layers The host's numerous skin layers is where the tick burrows to complete its feeding cycle.

{20} Palps Located on either side of the barbed hypostome (ref #18), the palps serve as protection for the tick's sensitive cheliceral digits (ref #17) and barbed hypostome (ref #18). The palps relax to either side of the body during feeding lying parallel to the host's dermis (ref #19). During actuation of Medically Inspired Tick Removal Instrument, the palps stiffen indicating the purposeful relaxation of the buried, barbed hypostome (ref #18) easing complete removal.

{21} Anterior Spurs Located at the base of each of the palps (ref #20), the anterior spurs are positioned by the palps into the host's dermis (ref #19). As the purposeful relaxations of the palps (ref #20) occur, the anterior spurs are pulled away from the host, dislodging them from the dermis (ref #19). This further eases the removal of the tick from the host.

{22} Alloscutum The alloscutum is located in the dorsal region of the tick. It is located just posterior to the anterior spurs (ref #21). It is the most anterior, recognizable body section in the podosoma (ref #16). Only the hard-bodied tick classes such as the Ixodes have a discernible alloscutum. The alloscutum is completely penetrated by the actuation of Medically Inspired Tick Removal Instrument's hemispherical protruding ball (ref #7).

{23} Cement The tick releases an agent in the wound to block coagulation. The tick secretes a sticky substance often referred to as cement which effectively adheres the cheliceral digits (ref #17) and barbed hypostome (ref #18) in place. When the mouthparts become detached from the rest of the body, they are especially problematic to remove. The only recourse is to wait for the host's body to react to the foreign item, swell and begin dispensing the foreign body.

1. Medically Inspired Tick Removal Instrument is a tool created for the express purpose of removing ticks completely, to include their cheliceral digits and barbed hypostome, from their host's dermis without promoting the transmission of harmful fluids from within the tick into the victim.
The tick is not squeezed during the removal process; the regurgitation of body fluids which may contain harmful bacteria or diseases is mitigated.
The closure of Medically Inspired Tick Removal Instrument effectively paralyzes the tick by application of the hemispherical protruding ball upon the back of the tick's alloscutum.
This paralyzing effect is evident by the sudden straightening of the tick's front legs.
The pinning action of the actuation of Medically Inspired Tick Removal Instrument forces the tick to release its grip.
